(19) United States
(12) Patent Application Publication
(10) Pub. No.: US 2026/0354964 A1
(43) Pub. Date: Nov. 12, 2026

(54) HIGH REFRACTIVE INDEX, HIGH ABBE NUMBER INTRAOCULAR LENS MATERIALS

(71) Applicant: Alcon Inc., Fribourg (CH)

(72) Inventors: Thomas Joseph Martin, Mansfield, TX (US); Douglas Schlueter, Azle, TX (US); Christine Anne Mathew, Fort Worth, TX (US); Ali Akinay, Berkeley Heights, NJ (US)

(21) Appl. No.: 19/660,607

(22) Filed: Apr. 28, 2026

Related U.S. Application Data
(60) Provisional application No. 63/796,510, filed on Apr. 29, 2025.

(51) Int. Cl. A61F 2/16; C08F 220/10

(52) U.S. Cl. CPC A61F 2/16; C08F 220/10; A61F 2002/16965

(57) ABSTRACT

High refractive index, high Abbe number, hydrophobic, acrylic materials are disclosed herein. In particular, these materials have both a high refractive index and a high Abbe number. This combination yields materials with a low refractive dispersion and chromatic aberration, and are therefore particularly suited for use as intraocular lens materials.

### FIELD

The present disclosure is directed to acrylic device materials. In particular, the present disclosure relates to high refractive index, high Abbe number acrylic device materials particularly suited for use as intraocular lens (IOL) materials, which can be injected through small incisions of less than 2.5 mm.

### BACKGROUND

With the recent advances in small-incision cataract surgery, increased emphasis has been placed on developing soft, foldable materials suitable for use in artificial lenses. In general, these materials fall into one of three categories: hydrogels, silicones, and acrylics.

In general, hydrogel materials have a relatively low refractive index, making them less desirable than other materials because of the thicker lens optic necessary to achieve a given refractive power. Silicone materials generally have a higher refractive index than hydrogels, but tend to unfold quickly after being placed in the eye in a folded position. Premature unfolding can potentially damage the corneal endothelium and/or rupture the natural lens capsule. Acrylic materials are desirable because they typically have a higher refractive index than silicone materials and unfold more slowly or controllably than silicone materials.

U.S. Pat. No. 5,290,892 discloses high refractive index, acrylic materials suitable for use as an IOL material. These acrylic materials contain, as principal components, two aryl acrylic monomers. They also contain a cross-linking component. The IOLs made of these acrylic materials can be rolled or folded for insertion through small incisions.

U.S. Pat. No. 5,331,073 also discloses soft acrylic IOL materials. These materials contain as principal components, two acrylic monomers which are defined by the properties of their respective homopolymers. The first monomer is defined as one in which its homopolymer has a refractive index of at least about 1.50. The second monomer is defined as one in which its homopolymer has a glass transition temperature less than about 22 °C. These IOL materials also contain a cross-linking component. Additionally, these materials may optionally contain a fourth constituent, different from the first three constituents, which is derived from a hydrophilic monomer. These materials preferably have a total of less than about 15% by weight of a hydrophilic component.

U.S. Pat. No. 5,693,095 discloses foldable ophthalmic lens materials comprising a total of at least 90% by weight of only two principal lens-forming monomers. One lens-forming monomer is an aryl acrylic hydrophobic monomer. The other lens-forming monomer is a hydrophilic monomer. The lens materials also comprise a cross-linking monomer and optionally comprise a UV absorber, polymerization initiators, reactive UV absorbers and reactive blue-light absorbers.

U.S. Pat. No. 6,653,422 discloses foldable ophthalmic lens materials consisting essentially of a single device-forming monomer and at least one cross-linking monomer. The materials optionally contain a reactive UV absorber and optionally contain a reactive blue-light absorber. The single device-forming monomer is present in an amount of at least about 80% by weight. The device-forming monomer is an aryl acrylic hydrophobic monomer.

Acrylic materials with a higher refractive index have historically been preferred as IOL materials because thinner lenses can be made for a given dioptric power. Thus, a lens made from a higher refractive index material may be implanted through a smaller incision than a similar power lens made from a lower refractive index material. Use of a smaller incision in turn results in fewer traumas and reduces the likelihood of surgically induced astigmatism. However, polymer materials with a high refractive index also generally exhibit refractive dispersion. This can result in chromatic aberrations that may impact visual performance of a lens when viewing light of differing wavelengths.

For example, the presence of aromatic groups tends to give materials with higher refractive dispersion. Hydrophobic acrylic materials without aromatic groups may have reduced refractive dispersion but will also have lower refractive index, thus will require a larger incision size than a comparable lens made from a high refractive index polymer.

Further limitations and disadvantages of conventional and traditional approaches will become apparent to one of skill in the art, through comparison of such systems with the present disclosure as set forth in the remainder of the present application with reference to the drawings.

### SUMMARY

In disclosed examples, an ophthalmic device material can be made by polymerizing a mixture of monomers, the mixture including: a) 65-75 wt. % of a first hydrophobic monomer; b) 15-17 wt. % of a hydrophilic monomer; c) 5-15 wt. % of a second hydrophobic monomer; and d) 1.5-3 wt. % of a cross-linking agent having a molecular weight of 100-500 Daltons; wherein the ophthalmic device material has a refractive index of 1.48-1.50, an Abbe number greater than 52, a Tg<15 °C, and an equilibrium water content less than 2%.

In some disclosed examples, an ophthalmic device material can be made by polymerizing a mixture of monomers, the mixture including: a) 2-cyclohexylethyl acrylate; b) 2-hydroxethyl methacrylate; c) N-octyl acrylate; and d) a cross-linking agent having a molecular weight of 100-500 Daltons; wherein the ophthalmic device material has a refractive index of 1.48-1.50, an Abbe number greater than 52, a Tg<15 °C, and an equilibrium water content less than 2%.

In some disclosed examples, ophthalmic device material can be made by polymerizing a mixture of monomers, the mixture including: a) 77-82 wt. % 2-cyclohexylethyl acrylate; b) 16-19 wt. % 2-hydroxyethyl methacrylate; c) 5-15 wt. % N-octyl acrylate; and d) 1.4-1.8 wt. % neopentyl glycol diacrylate; wherein the ophthalmic device material has a refractive index of 1.48-1.50, an Abbe number greater than 50, and a Tg<10 °C.

These and various other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 illustrates an example table of candidate components for acrylic device materials, in accordance with an example embodiment of the disclosure.

FIG. 2 illustrates another example table of candidate components for acrylic device materials, in accordance with an example embodiment of the disclosure.

FIG. 3 Table of candidate components for formulation examples 1-3, in accordance with an example embodiment of the disclosure.

FIG. 4 illustrates an example table of representative results of the evaluated example acrylic device materials of FIG. 3, in accordance with an example embodiment of the disclosure.

FIG. 5 Table of candidate components for formulation examples 4-5, in accordance with an example embodiment of the disclosure.

FIG. 6 illustrates an example table of representative results of the evaluated example acrylic device materials of FIG. 5, in accordance with an example embodiment of the disclosure.

FIG. 7 Table of candidate components for formulation examples 6-9, in accordance with an example embodiment of the disclosure.

FIG. 8 illustrates an example table of representative results of the evaluated example acrylic device materials of FIG. 7, in accordance with an example embodiment of the disclosure.

FIG. 9 Table of candidate components for formulation examples 10-14, in accordance with an example embodiment of the disclosure.

FIG. 10 illustrates an example table of representative results of the evaluated example acrylic device materials of FIG. 8, in accordance with an example embodiment of the disclosure.

FIG. 11 Table of candidate components for formulation examples 15-18, in accordance with an example embodiment of the disclosure.

FIG. 12 illustrates an example table of representative results of the evaluated example acrylic device materials of FIG. 11, in accordance with an example embodiment of the disclosure.

FIG. 13 Table of candidate components for formulation examples 19-21, in accordance with an example embodiment of the disclosure.

FIG. 14 illustrates an example table of representative results of the evaluated example acrylic device materials of FIG. 13, in accordance with an example embodiment of the disclosure.

FIGS. 15A and 15B illustrate example Abbe refractometer test images of a lens employing the disclosed acrylic device materials and another lens, respectively, in accordance with an example embodiment of the disclosure.

FIG. 16 illustrates example Equations 1 and 2, in accordance with an example embodiment of the disclosure, used to determine Abbe number (ν_{D}) and equilibrium water content (% EWC), respectively.

FIGS. 17A and 17B illustrate example chemical structures for first and second hydrophobic monomers, respectively, in accordance with example hydrophobic monomer embodiments of the disclosure.

FIGS. 17C and 17D illustrate example general chemical structures III and IV, respectively, in accordance with example hydrophilic monomer embodiments of the disclosure.

The figures are not necessarily to scale. Where appropriate, similar or identical reference numbers are used to refer to similar or identical components.

### DETAILED DESCRIPTION

Improved soft yet durable, foldable acrylic materials which are particularly suited for use as IOLs, but which are also useful as other implantable ophthalmic devices, such as keratoprostheses, corneal rings, corneal implants, and corneal inlays are disclosed herein. Such foldable acrylic materials and the resulting lenses have both high refractive index and high Abbe number. The high refractive index and high Abbe number are accomplished using a combination of monomers, including two or more monomers containing aliphatic functional groups in a hydrophobic acrylic polymer. The foldable acrylic materials of the present disclosure include copolymers formed by polymerizing a mixture of monomers comprising a major amount of first and second hydrophobic acrylic monomers, a hydrophilic monomer, and a cross-linking agent.

The implantable ophthalmic device materials disclosed herein are optically clear, such that they are suitable for use as IOLs, and they have low tack, low surface scatter, good stability profile and good delivery properties. Among other factors, the presently disclosed materials and lenses are based on the finding that a multi-component, copolymeric, high refractive index and high Abbe device material, obtained by copolymerizing the ingredients mentioned herein, is soft, durable, glistening-free, has low tack and low haze, has low surface light scatter, and is capable of implantation through small (2.5 mm or less) incisions with good unfolding properties.

Cataracts impact the vision of millions of people by clouding the natural lens, often progressing over a number of years. Often, vision can be corrected with implantation of an IOL. Although IOL surgery has been successfully used for many years for many patients, a still unmet need in cataract replacement surgery is due to the potential for the appearance of a crescent shadow at the periphery of a patient's visual field post-implantation. Though relatively rare, this phenomenon can be persistent and bothersome to some patients, which may later require an explant of the implanted IOL. This phenomenon, known as negative dysphotopsia, may be mitigated by increasing the diameter of the IOL to redirect light on to the retina that may otherwise miss the optic body. However, the larger optic tends to translate to a larger volume IOL. The larger optic may create challenges for a successful implantation of the larger volume IOL, as the larger volume IOL will have a larger center thickness and still pass through a small incision (e.g., approximately 2.2 mm) via a delivery device.

There is also a desire to reduce chromatic aberration (or dispersion) induced by some IOL materials. For example, reducing a refractive index (RI, or n) of materials used to form IOLs can have this effect. A low refractive index material is typically associated with a larger volume IOL, as a lower refractive index results in an increased radius of curvature and center thickness to achieve a dioptric power similar to IOLs formed of higher RI materials. However, conventional acrylic materials created with relatively high refractive index (e.g., approximately 1.5 RI) are difficult to create while maintaining low chromatic dispersion.

As disclosed herein, materials are provided exhibiting low chromatic dispersion, while maintaining a sufficiently low modulus and high strain at break able to withstand higher compression forces without damage to the lens during small incision delivery into a patient's capsular bag. This is accomplished by creating a material comprising one or more monomers, such as those containing aliphatic functional groups, in an acrylic polymer (e.g., a hydrophobic acrylic polymer), utilizing a relatively low concentration of cross-linker.

Polymer materials with both high refractive index and high Abbe number are considered as optical materials due to favorable performance characteristics in comparison to inorganic glasses. Selection of constituent components leverages a known relationship between refractive index (e.g., n_{D}) and Abbe number (ν_{D}) of candidates.

For example, materials with a high refractive index generally have a low Abbe number. That is, there is a significant difference between the refractive index of blue light (n_{F}) and the refractive index of red light (n_{C}) of a given material. Example materials developed for spectacle lens applications include polymethyl methacrylate, polycarbonate, cycloolefins, episulfides, polythiols, and/or polyisocyanates, as a list of non-limiting examples. However, these materials have generally proven unsuitable for small incision IOL implantation due to inflexible mechanical properties and/or long term stability profiles after surgery.

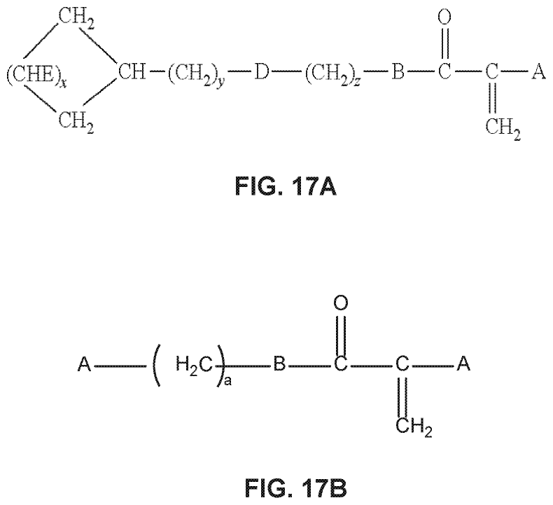

As employed herein, the Abbe number of a material is a measure of its chromatic aberration (or dispersion). It can be used for calculating the focal length mismatch of a lens over the visible spectral range, and is used for classifying materials according to the Abbe diagram. The Abbe number of a transparent optical material is defined by Equation 1 as presented in FIG. 16:

$${v}_{D} = \frac{{n}_{D} - 1}{{n}_{F} - {n}_{C}} \qquad ( Equation 1 )$$

As provided in Equation 1, n_{D}, n_{F}, and n_{C} are the refractive indices of the material at 589 nm (sodium D), 486 nm (hydrogen F), and 656 nm (hydrogen C), respectively. The Abbe number definition considers refractive indices at three different, standard wavelengths in the visible region: λ_{F}=486.1 nm (blue Fraunhofer F line from hydrogen); λ_{D}=589.2 nm (orange Fraunhofer D line from sodium); λ_{C}=656.3 nm (red Fraunhofer C line from hydrogen).

The human eye is particularly sensitive to the above wavelengths. The measure of chromatic dispersion in the resulting material used in an IOL can provide an indication of the visual clarity of a particular lens.

As employed herein, chromatic dispersion refers to the change in refractive index with frequency of incident light. For example, the chromatic dispersion of an optical material is the phenomenon that the phase velocity and group velocity of light propagating in a transparent medium depend on the optical frequency.

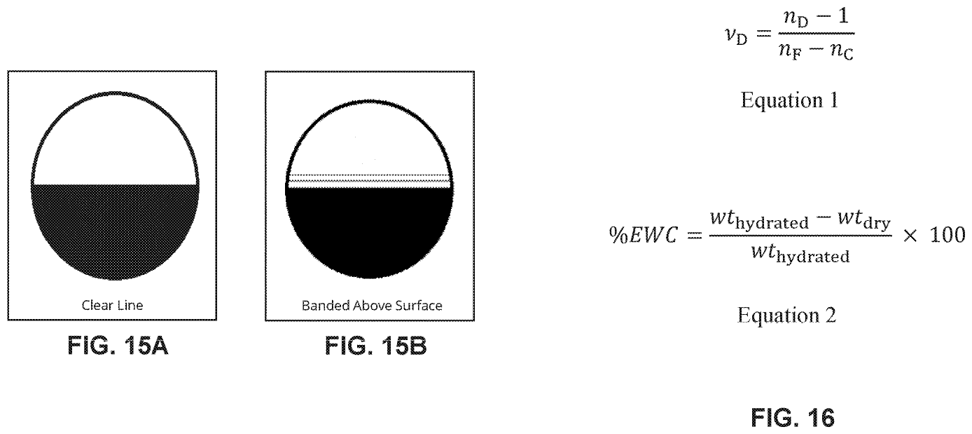

A preferred lens material will have a homogeneous RI between the surface and the bulk, represented in FIGS. 15A and 15B. A clear line implies surface to bulk homogeneity while a banded line implies a difference in RI between the surface and the bulk. A subjective rating of 1-4 is applied, where 1 represents a clear line and 2-4 represent degrees of banding.

As employed herein, glistening is defined as vacuoles that can develop within a lens. Such vacuoles can occur in a variety of materials, but are often associated with hydrophobic acrylic materials. Glistening can impact visual quality by, for example, increasing intraocular light scattering, observed as glare. Advantageously, the disclosed materials exhibit reduced or unmeasurable glistening, resulting in clearer vision. Further, opacification is less noticeable or unmeasurable, such that haze, or cloudiness, has little impact on vision through the lens.

As disclosed herein, materials have been developed to overcome the challenges of conventional materials. These example materials result in a higher Abbe number and exhibit improved optical properties due in part to a reduction in chromatic aberration compared to materials with lower Abbe number. Reduction in glass transition number (T_{g}) below operating room temperatures will also improve the consistency of unfolding behavior and material properties at operating room temperatures (e.g., about 18 °C to 23 °C). Tg can be below about 15 °C, and more preferably below about 10 °C. Tg is measured by differential scanning calorimetry at 10 °C/min., and is determined as the half-height of the heat capacity increase. The lower modulus and greater elongation at break (based on tensile testing) also allows for small incision delivery even while employing a larger volume IOL design and is accomplished without compromising other desirable IOL characteristics, such as material clarity or stability profile over the life of the product.

In accordance with this disclosure, a first hydrophobic monomer may be used in the synthesis of hydrophobic acrylic polymers included in IOLs of the present disclosure. In some embodiments, the first hydrophobic monomer is a cycloaliphatic acrylic monomer of general formula (I) as shown below:

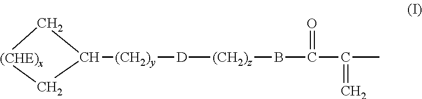

wherein: A is H, CH₂CH₃, or CH₃;
B is O, NR, or S;
D is O, S, or a single bond;
E is CH₃, CH₂CH₃, CH(CH₃)₂, C(CH₃)₃, CH₂OH or H;
R is H, CH₃, CH₂CH₃, or CH(CH₃)₂, H or a branched or linear C1-C10 alkyl;
x is an integer of 1-4, such as 1, 2, 3, or 4, provided that if x is >1, then not more than one CHE group has E≠H;
y is an integer of 0-2, such as 0, 1, or 2; and
z is an integer of 0-4, such a 0, 1, 2, 3, or 4, provided that if D=a single bond, then z≠0.

In some embodiments, acrylic hydrophobic monomers for use in the materials of the present disclosure are those of formula (I) wherein B is O, z is 0-2, D is O or a single bond, y is 0, x is 2 or 3, and E is independently H, CH₂OH or CH₃, provided if z=0 or 1, then D is a single bond. Most preferred is B is O, z is 2, D a single bond, y is 0, x is 3, and E is H. For example, monomers can include 2-cyclohexylethyl acrylate, 2-cyclopentylethyl acrylate, 3-cyclohexylpropyl acrylate, 3-cyclopentylpropyl acrylate and 2-(cyclohexyloxy)ethyl acrylate. Most preferred is 2-cyclohexylethyl acrylate.

In accordance with the disclosure, a second hydrophobic monomer may be used in the synthesis of hydrophobic acrylic polymers. In some embodiments, the second hydrophobic monomer is an aliphatic acrylic monomer of general formula (II) as shown below:

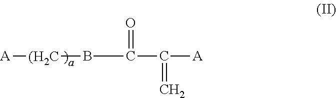

wherein: each A is independently H, CH₂CH₃, or CH₃;
B is O, NR, or S;
R is H, CH₃, CH₂CH₃, or CH(CH₃)₂, or a branched or linear C1-C10 alkyl; and
a is an integer of 2-12, such as 2, 3, 4, 5, 6, 7, 8, 9, 10, 11, or 12.

In some embodiments, acrylic hydrophobic monomers for use in the materials of the present disclosure are those of formula (II) wherein A is H or CH₃; B is O; and a is 4-10. For example, monomers can include n-butyl acrylate (BA), n-hexyl acrylate (HA), n-octyl acrylate (nOA), n-decyl acrylate and/or n-dodecyl acrylate (DDA). Most preferred is n-octyl acrylate (nOA). In some embodiments, R contains a branched alkyl, as present in 2-ethylhexyl acrylate, sec-butyl acrylate, isopropyl acrylate, 3-pentyl acrylate, or neopentyl acrylate.

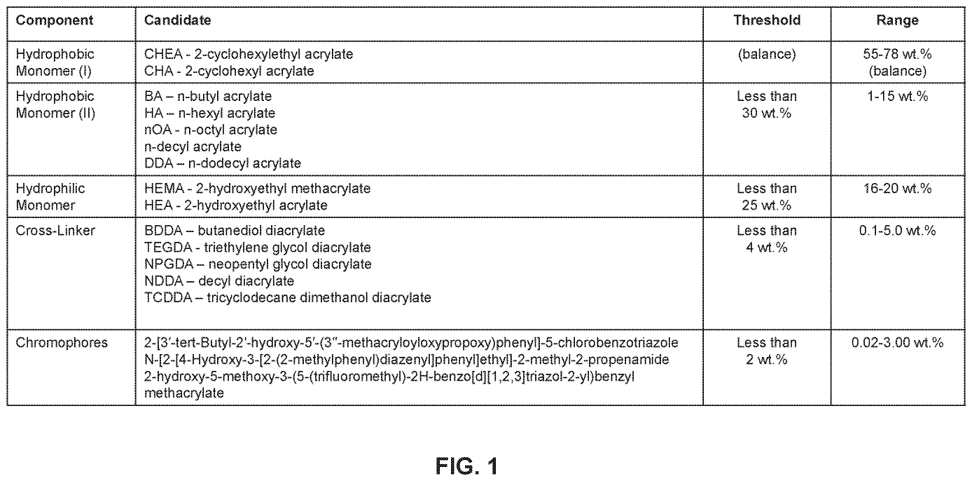

FIG. 1 provides a table listing components used to create a material for IOL with the desired characteristics described herein. As shown, first and second hydrophobic monomers can be combined with hydrophilic monomers, cross-linker agents, and one or more additional components (such as chromophores). As provided, the composition used to form the IOL can contain a first hydrophobic monomer (I)—with a primary function of enhancing optical function of the material—which can include one or more cycloaliphatic acrylic monomers of general formula (I) as shown above, such as 2-cyclohexylethyl acrylate (CHEA) and/or 2-cyclohexyl acrylate (CHA), at a concentration ranging from 55-78% by weight, but could range from 50-90%. In some additional or alternative examples, the first hydrophobic monomer can include 2-cyclopentylethyl acrylate, 3-cyclohexylpropyl acrylate, 3-cyclopentylpropyl acrylate, 2-(cyclohexyloxy)ethyl acrylate, or a combination thereof. All formula concentration values refer to the formulation feed composition. These concentration values may not match the final copolymer composition as bulk copolymerization typically does not achieve 100% conversion.

One or more second hydrophobic monomer (II)—with a primary function of enhancing mechanical function of the material—can include one or more aliphatic acrylic monomers of general formula (II) as shown above such as n-butyl acrylate (BA), n-hexyl acrylate (HA), n-octyl acrylate (nOA), n-decyl acrylate, and/or n-dodecyl acrylate (DDA) at a concentration ranging from 1-15% by weight, but could range from 2-25%. (e.g., preferably less than 30% by weight).

First and second hydrophobic monomers can be made by known methods. For example, the conjugate alcohol of the desired monomer can be combined in a reaction vessel with methyl acrylate, tetrabutyl titanate (catalyst), and a polymerization inhibitor such as 4-benzyloxy phenol. The vessel can then be heated to facilitate the reaction and distill off the reaction by-products to drive the reaction to completion. Alternative synthesis schemes involve adding acrylic acid to the conjugate alcohol and catalyzing with a carbodiimide or mixing the conjugate alcohol with acryloyl chloride and a HCl acceptor such as pyridine or triethylamine.

A hydrophilic monomer—with a primary function of enhancing clarity of the material—can include acrylate or methacrylate monomers having hydroxyl side chains, such as one or more of 2-hydroxyethyl methacrylate (HEMA) and/or 2-hydroxyethyl acrylate (HEA) at a concentration ranging from 16-20% by weight of the composition to form the IOL, but could range from 10-25% (e.g., preferably less than 25% by weight). For example, general formula (III) where A is H, CH₃ or CH₂CH₃; B is O, NR, or S; R is H, CH₃, CH₂CH₃, or CH(CH₃)₂, H or a branched or linear C1-C10 alkyl; p is an integer 0-6; and general formula (IV) where A is H, CH₃ or CH₂CH₃; B is O, NR, or S; R is H, CH₃, CH₂CH₃, or CH(CH₃)₂, H or a branched or linear C1-C10 alkyl; E is H or CH₃; q is an integer 1-25.

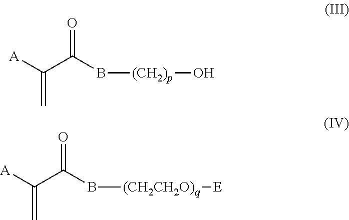

The copolymer materials of the present disclosure are cross-linked. The copolymerizable cross-linking agent used in the copolymers may be any unsaturated compound having more than one unsaturated group (e.g., a terminally ethylenically unsaturated compound). Suitable cross-linking agents include, for example low molecular weight cross-linking agents having a molecular weight from 100-500 Daltons, including butanediol diacrylate (BDDA), triethylene glycol diacrylate (TEGDA), neopentyl glycol diacrylate (NPGDA), decyl diacrylate (NDDA), and/or tricyclodecane dimethanol diacrylate (TCDDA), and their corresponding methacrylates. The cross-linkers can be included at a concentration ranging from 0.1-5.0% by weight (e.g., preferably less than 4% by weight) in the IOL formulation.

In some additional or alternative examples, the cross-linking monomer can include one or more of ethylene glycol diacrylate; diethylene glycol diacrylate; allyl acrylate; 1,3-propanediol diacrylate; 2,3-propanediol diacrylate; 1,6-hexanediol diacrylate; cyclohexane-1,1-diyldimethanol diacrylate, 1,4-cyclohexanediol diacrylate, 1,3-adamantanediol diacrylate, 1,3-adamantanedimethyl diacrylate, 2,2-diethyl-1,3-propanediol diacrylate, 2,2-diisobutyl-1,3-propanediol diacrylate, 1,3-cyclohexanedimethyl diacrylate, or 1,4-cyclohexanedimethyl diacrylate.

Additives, such as chromophores, can be included in IOLs of the present disclosure. In some embodiments, chromophores include reactive (polymerizable) blue light blockers, for example N-[2-[4-hydroxy-3-[2-(2-methylphenyl)diazenyl]phenyl]ethyl]-2-methyl-2-propenamide or chromophores that block both blue and ultraviolet light (e.g., 2-[3′-tert-butyl-2′-hydroxy-5′-(3″-methacryloyloxypropoxy)phenyl]-5-chlorobenzotriazole, 2-hydroxy-5-methoxy-3-(5-(trifluoromethyl)-2H-benzo[d][1,2,3]triazol-2-yl)benzyl methacrylate, or combinations thereof), at a concentration ranging from 0.02-3.00% by weight (e.g., such as less than 2% by weight).

Additives such as reactive UV light absorbers that only block light <400 nm can be included in IOLs of the present disclosure. For example, UV light absorbers can include 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, 2-(3-(tert-butyl)-4-hydroxy-5-(5-methoxy-2H-benzo[d][1,2,3]triazol-2-yl)phenoxy)ethyl methacrylate, or combinations thereof. UV absorbers are typically present in an amount from 0-5 wt. %, but can contain 0-2 wt. %, of a reactive UV absorber, where the wt. % reflects the composition to form the IOL.

Additives such as reactive blue-light absorbing compounds can be included in IOLs of the present disclosure. In some embodiments, a blue-light absorbing dye is N-2-[3-(2′-methylphenylazo)-4-hydroxyphenyl]ethyl methacrylamide. When present, blue-light absorbers may be used in an amount from 0.005-2 wt. %, or 0.01-0.10 wt. % of the composition to form the IOL.

Representative reactive blue-light absorbing compounds are described in U.S. Pat. Nos. 5,470,932; 8,207,244; and 8,329,775. For example, U.S. Pat. No. 5,470,932 relates to polymeric yellow dyes of the azo family in “blue-blocking” for use in ophthalmic lenses. In particular, the polymerizable yellow dyes are described as soluble in organic monomers, such as acrylic/methacrylic monomers, and contain in their chemical structure one or more acrylic or methacrylic functional groups which are reactive towards free radical polymerization. These dyes, when polymerized with organic monomers capable of forming a transparent material, will be bonded to the polymer and thus greatly reduce the amount of dye which can leach out of the material. As a result, these dyes can be used in transparent materials to decrease the intensity of blue light transmitted through them. These transparent materials with one or more of the bondable yellow dyes incorporated in them may be extracted with organic solvents to remove unreacted monomers, low molecular weight oligomers and low molecular weight polymers, as well as other impurities, and then used to make ocular lenses such as intraocular lenses (IOLs), contact lenses, eyeglasses and other windows. These transparent materials containing yellow dye may also be used to make lens coating materials.

U.S. Pat. No. 8,207,244 describes azo compounds that contain reactive groups, which allow for covalent attachment of the absorbers to ocular lens materials. For example, the described azo compounds are suitable for use in ophthalmic device materials, such as IOLs. The device materials containing an azo compound can also contain a UV absorber and/or other visible light absorber. Many reactive (copolymerizable) UV absorbers suitable for use in implantable ophthalmic lenses and devices are described herein. UV absorbers are often present in IOL materials, as disclosed herein.

U.S. Pat. No. 8,329,775 is directed to visible light absorbers, such as azo compound monomers suitable for use in IOL materials. These azo compounds are suitable for use as monomers that absorb a portion of visible light (approximately 380-495 nm). These absorbers may be employed in ophthalmic lenses, including contact lenses. Such azo compounds contain reactive groups, which allow for covalent attachment of the absorbers to ocular lens materials. Advantageously, the absorbers can be synthesized in a limited number of steps from readily available, inexpensive starting materials without the need for column chromatography.

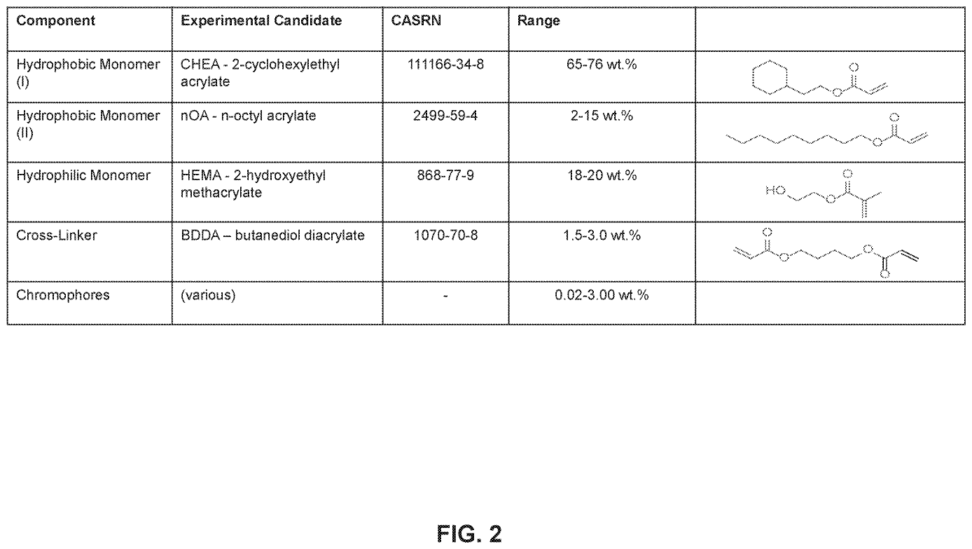

FIG. 2 provides a table listing an example composition of a material suitable for IOL implantation while exhibiting desirable characteristics of an IOL. As shown, 2-cyclohexylethyl acrylate (CHEA) is selected as the first hydrophobic monomer (I), and n-octyl acrylate (nOA) is selected as the second hydrophobic monomer (II). Hydrophilic monomer selection is 2-hydroxyethyl methacrylate (HEMA), the selected cross-linker is butanediol diacrylate (BDDA), and one or more chromophores are selected to include blue blockers and ultraviolet blockers (e.g., 2-[3′-tert-Butyl-2′-hydroxy-5′-(3″-methacryloyloxypropoxy)phenyl]-5-chlorobenzotriazole, N-[2-[4-hydroxy-3-[2-(2-methylphenyl)diazenyl]phenyl]ethyl]-2-methyl-2-propenamide, 2-hydroxy-5-methoxy-3-(5-(trifluoromethyl)-2H-benzo[d][1,2,3]triazol-2-yl)benzyl methacrylate, or combinations thereof). In this example, CHEA is selected to enhance material flexibility, exhibiting a high RI and high Abbe number. nOA is selected to enhance material flexibility by reducing modulus of the material. HEMA is a glistening resistant additive. BDDA (and other cross-linkers, such as TEGDA, NDDA) are selected to improve material strength and preserve lens shape stability.

The monomer mixture that is polymerized to form the ophthalmic device materials contains first and second hydrophobic monomers, a hydrophilic monomer, a cross-linking agent, and may contain one or more additives, such as a UV absorber and/or a blue-light absorber. In some embodiments, the monomer mixture is substantially or completely free of an aromatic monomer (such as phenylethyl acrylate), such as less than 1% by weight, such as 0% by weight of aromatic monomer.

### Examples 1-3

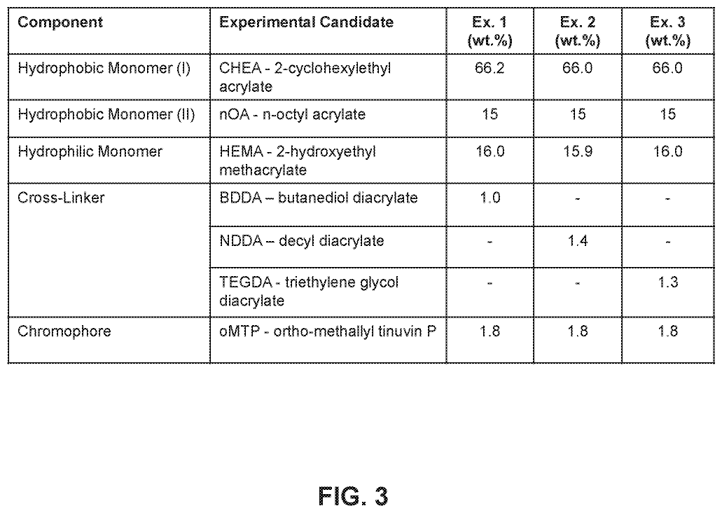

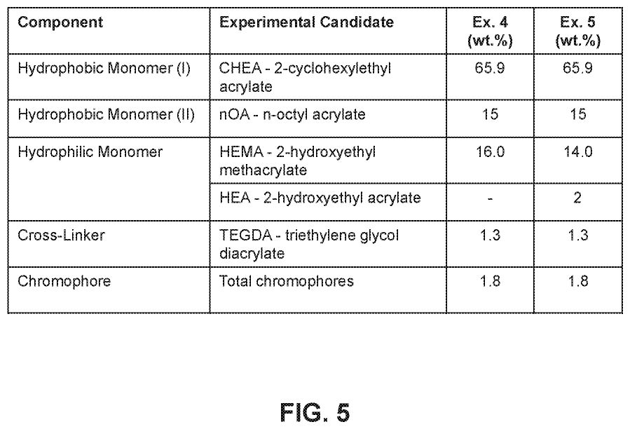

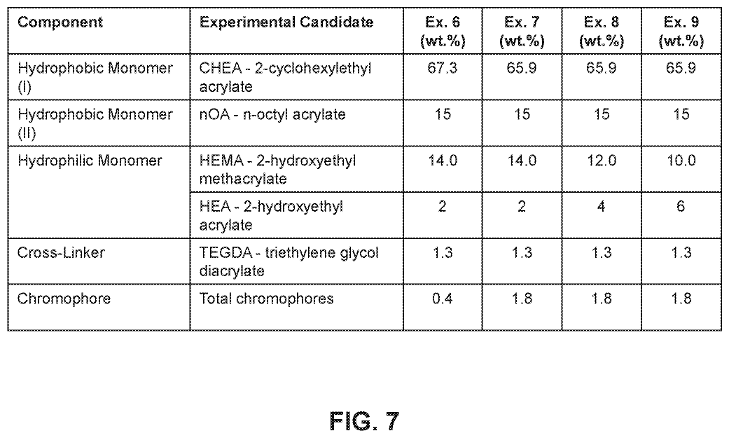

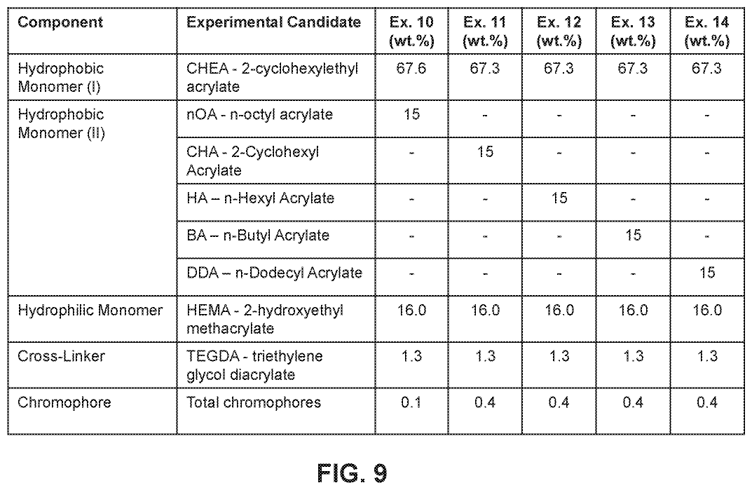

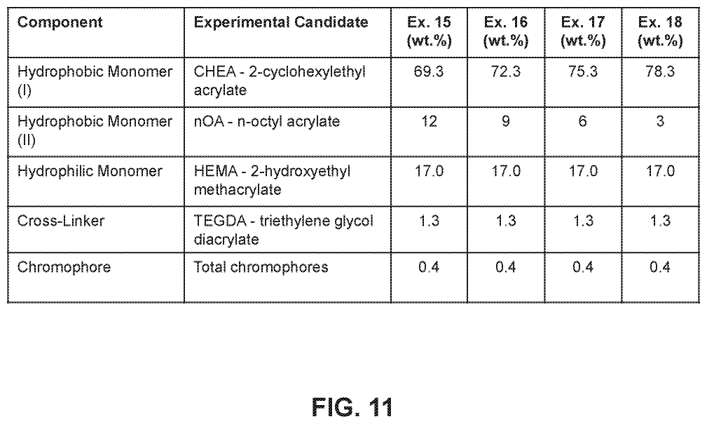

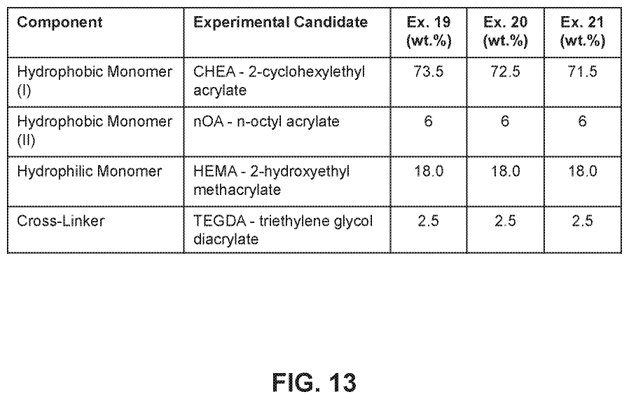

A monomer solution was prepared by combining components in proportions as listed in the tables provided in FIGS. 3, 5, 7, 9, 11, and 13. The materials represented by Examples 1-18, and Examples 19-21 and tier constituent amounts are provided in FIGS. 3, 5, 7, 9, 11, and 13. The monomer solution(s) were filtered through a 2.7 μm, 1.0 μm and 0.2 μm membrane stack, and then pipetted into lens molds or rectangular flat molds. The filled molds were placed in an oven, preheated to 95 °C for 3 hours. The molds were allowed to cool to 30 °C before removing them from the oven. Once cooled to the desired temperature, the product was removed from the molds, extracted in acetone at ambient temperature (e.g., 18-22 °C) with multiple exchanges (e.g., 6×), and allowed to air dry for a period of time (e.g., 12 hours). The product was then placed in a vacuum oven at 80 °C for a 16 hour cycle. Tensile properties were determined using an Instron material tester (such as Instron Material Tester Model No. 4442) with a 50 N load cell using a crosshead speed of 41 mm/min. Refractive index and Abbe number of the product was measured using an ATAGO DR-M2 multi-wavelength Abbe refractometer. Samples were equilibrated in a balanced salt solution (BSS) at 35 °C prior to measurement. The Abbe number (ν_{D}) was calculated using Equation 1:

$${v}_{D} = \frac{{n}_{D} - 1}{{n}_{F} - {n}_{C}} \qquad ( Equation 1 )$$

where n_{D}, n_{F}, and n_{C} are the refractive indices of the material at 589 nm (sodium D), 486 nm (hydrogen F), and 656 nm (hydrogen C), respectively.

The equilibrium water content (EWC) was gravimetrically determined using an analytical balance. First, the dry sample weight was obtained, then the sample was equilibrated in BSS at 35 °C for at least 24 hours. The sample was then removed from BSS, excess surface liquid was removed by blotting, and the sample was weighed. The percentage of EWC is then determined by use of the following Equation 2:

$$\% EWC = \frac{{wt}_{hydrated} - {wt}_{dry}}{{wt}_{hydrated}} \times 100 \qquad ( Equation 2 )$$

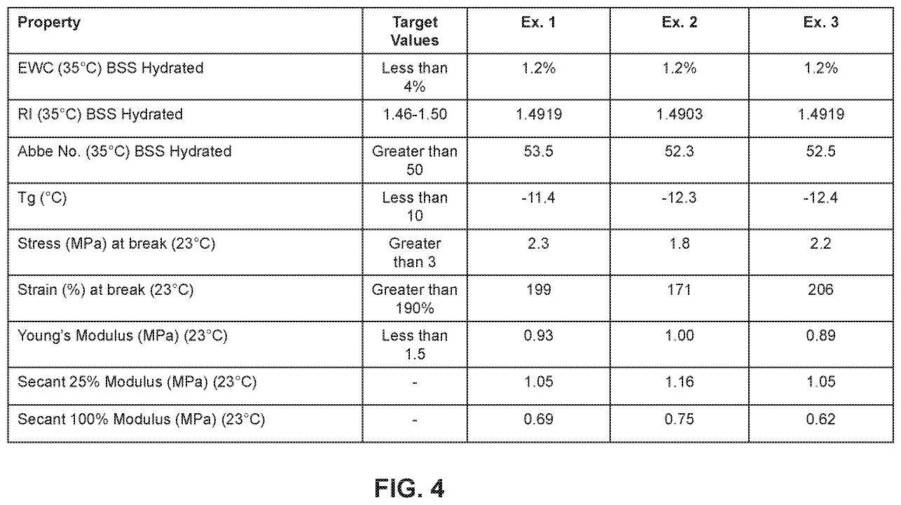

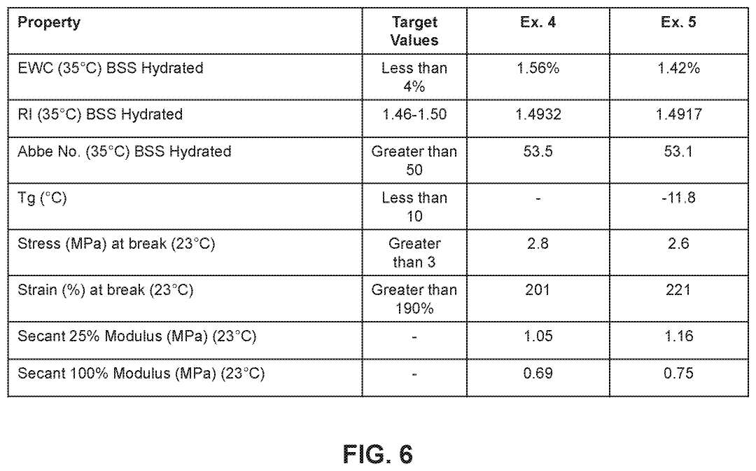

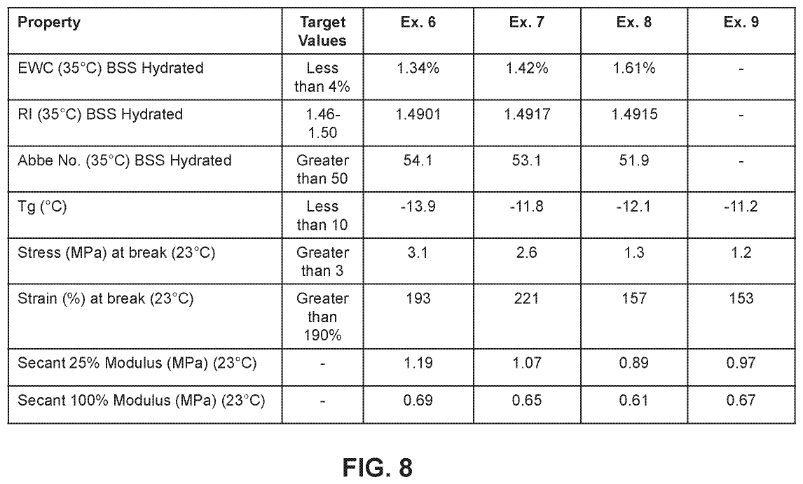

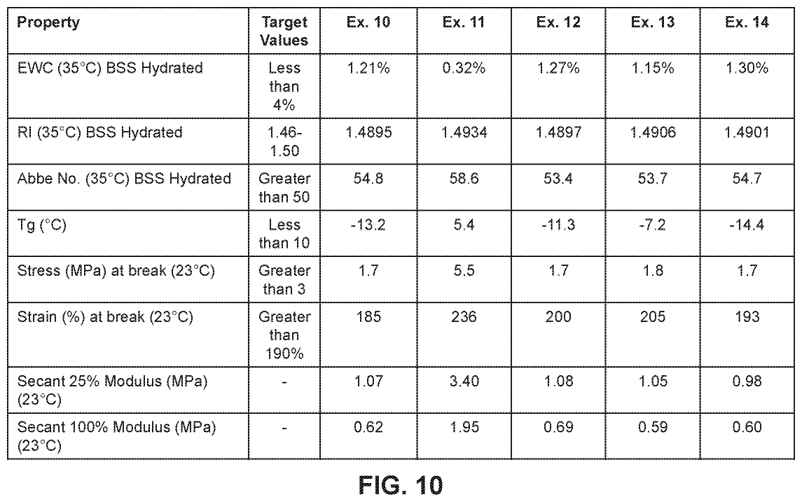

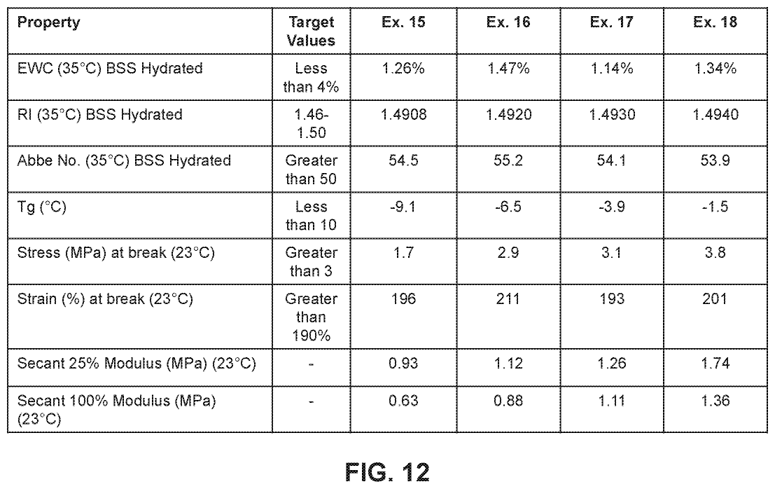

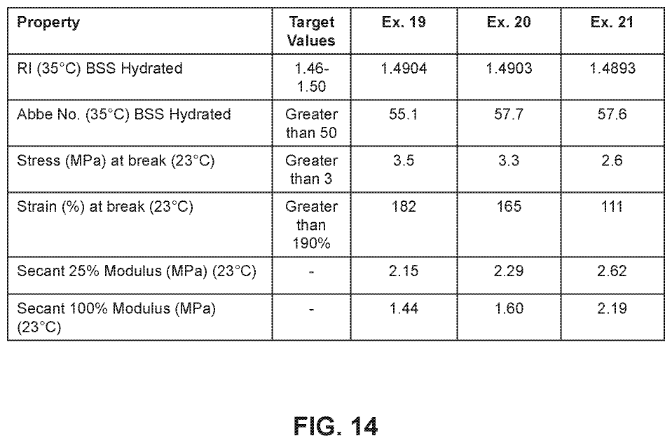

The results of this process are provided in the table shown in FIGS. 4, 6, 8, 10, 12, and 14. As shown, the use of nOA as a hydrophobic monomer yields results within a desired threshold and/or ranges of values.

For example, the lens material will have an EWC of less than 4%, preferably less than 2% once equilibrated in the patient's eye following implantation. In some examples, EWC can range from 0.1-4% and/or from 0.2-2%. The lens material may exhibit a refractive index of 1.46-1.50, preferably 1.48-1.50, and most preferably 1.49-1.50. Despite having a relatively high refractive index, the materials of the present invention have an Abbe number greater than 47, preferably greater than 50, and most preferably, greater than 52. Example Abbe number values for aliphatic acrylic polymers can be greater than 50, between 50 and 60 (calculated using Equation 1). In some examples, there could be little difference between n_{F} and n_{C}, therefore the Abbe number could be even larger. Refractive index and Abbe number are both measured using an Abbe refractometer and a material sample that has been equilibrated in balanced salt solution at 35 °C prior to measurement.

The glass-transition temperature (T_{g}) of the lens material, which reflects the material's ability to fold and unfold, is preferably below about 15 °C, and more preferably below about 10 °C. T_{g} is measured by differential scanning calorimetry at 10 °C/min., and is determined as the half-height of the heat capacity increase.

Stress at break is calculated at the maximum force for the sample, typically the force measured when the sample breaks, given the initial cross-sectional area. The stress is preferably above 3 MPa, and more preferably above 4 MPa (e.g., between 3-20 MPa). The lens material will have an elongation (strain at break) of at least 170%, preferably at least 180%, and most preferably at least 190%. This property indicates that the lens generally will not crack, tear or split when folded. Elongation of polymer samples is determined on dumbbell shaped tension test specimens with a 20 mm total length, overall width of 2.49 mm, 0.833 mm width of the narrow section, a fillet radius of 8.83 mm, and a thickness of about 0.9 mm. Testing is performed on samples at either 18 °C or 23 °C, and 35% relative humidity using a tensile tester. The grip distance is set at 9 mm and a crosshead speed is set at 41-mm/minute and the sample is pulled to failure. The strain at break is reported as the relative displacement at failure to the original grip distance. The Young's modulus is calculated from the instantaneous slope of the stress-strain curve in the linear elastic region, if present. The 25% secant modulus is calculated from the slope of a straight line drawn on the stress-strain curve between 0% strain and 25% strain. The 100% secant modulus is calculated from the slope of a straight line drawn on the stress-strain curve between 0% strain and 100% strain, the secant modulus being within a range of 0-6 MPa.

A glistening evaluation can be carried out by placing samples in deionized water at 45 °C for 20 hours. The samples are then transferred to a 37 °C. bath. Samples are inspected for glistenings after 2 hours of cooling to 37 °C using an optical microscope under dark field conditions with a magnification of at least 100×. The average number of glistenings per mm² in the sample is determined. Preferably, the materials of the present invention have less than 10 glistenings/mm², and more preferably less than 1 glistening/mm² (e.g., 0-10 glistenings/mm², preferably 0-1 glistenings/mm²).

The disclosed materials and IOLs exhibit desirable characteristics, such that demolding does not result in damage to the lens. Further, the surface of the lens is not excessively tacky, such that packaging and instruments are not unduly adhered to the IOL during installation. IOLs constructed of the materials disclosed herein can be of any design capable of being rolled or folded into a small cross section that can fit through a relatively smaller incision. For example, an IOL can be of what is known as a one piece or multipiece design, and comprise optic and haptic components. The optic is that portion which serves as the lens. The haptics are attached to the optic and hold the optic in its proper place in the eye. The optic and haptic(s) can be of the same or different material. A multipiece lens is so called because the optic and the haptic(s) are made separately and then the haptics are attached to the optic. In a single piece lens, the optic and the haptics are formed out of one piece of material. Depending on the material, the haptics are then cut, or lathed, out of the material to produce the IOL.

The fully processed IOL may have a lens size of approximately 6.5 mm. The IOL can be delivered through a small incision (e.g., 2.0 mm), involving a suitably flexible IOL (e.g., with a 100% secant modulus, less than about 3 MPa). As provided in FIGS. 4, 6, 8, 10, 12, and 14 the use of nOA (e.g., an 8-carbon monomer, an example of a straight chain aliphatic monomer) results in improved flexibility of the IOL.

The nOA can be replaced with an alternate side-chain length of differing carbon lengths, which can be one of C4, C6, C10 or C12. In some examples, suitable alternative configurations of C4, C6, C8, C10 and/or C12 mixtures may be employed as well. Although several examples are provided with (n-octyl acrylate) C8 as a preferred hydrophobic monomer, other chain length acrylate monomers were evaluated to determine the suitability of their resulting characteristics. Several such monomers (including but not limited to n-butyl acrylate, n-hexyl acrylate, 2-ethylhexyl acrylate, n-decyl acrylate and n-dodecyl acrylate) are identified as exhibiting reasonably well suited characteristics for the desired material.

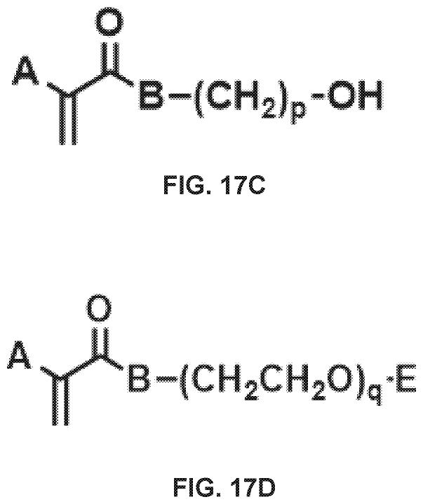

FIGS. 15A and 15B illustrate a preferred, clear line from the RI measurement of a lens formed of the disclosed optical materials with surface-to-bulk homogeneity, in contrast to a less homogeneous lens with a banded RI measurement result, respectively. FIG. 16 provides Equations 1 and 2. FIGS. 17A, 17B, 17C, and 17D illustrate example formulas for a first hydrophobic monomers, a second hydrophobic monomer, and general formulas III and IV, respectively. In particular, general formula III corresponds to A is H, CH₃ or CH₂CH₃; B is O, NR, or S; R is H, CH₃, CH₂CH₃, or CH(CH₃)₂, H or a branched or linear C1-C10 alkyl; p is an integer 0-6; and general formula (IV) where A is H, CH₃ or CH₂CH₃; B is O, NR, or S; R is H, CH₃, CH₂CH₃, or CH(CH₃)₂, H or a branched or linear C1-C10 alkyl; E is H or CH₃; q is an integer 1-25.

### EMBODIMENTS

The present disclosure is further directed to the following embodiments which may be combined with any embodiments described herein and/or may be isolated (optional) from any embodiments described herein.

Clause 1. An ophthalmic device material made by polymerizing a mixture of monomers wherein the mixture comprises:
a) 55-85 wt. % of a first hydrophobic monomer;
b) 10-22 wt. % of a hydrophilic monomer;
c) 2-18 wt. % of a second hydrophobic monomer; and
d) 1.0-4.0 wt. % of a cross-linking agent having a molecular weight of 100-500 Daltons;
wherein the ophthalmic device material has a refractive index of 1.48-1.50, an Abbe number greater than 52, a Tg<15 °C, and an equilibrium water content less than 2%.

Clause 2. The ophthalmic device material of Clause 1, wherein said first hydrophobic monomer is of cycloaliphatic acrylic monomer of formula (I):

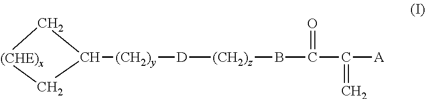

wherein: A is H or CH₃;
B is O, NR, or S;
D is O, S, or nothing (i.e., a single bond);
E is CH₃, CH₂CH₃, CH(CH₃)₂, C(CH₃)₃, CH₂OH or H;
R is H, CH₃, CH₂CH₃, or CH(CH₃)₂;
x is 1-4, provided that if x is >1, then not more than one CHE group has EH;
y is 0-2; and
z is 0-4, provided that if D=nothing, then z≠0.

Clause 3. The ophthalmic device material of Clauses 1 or 2 wherein for the cycloaliphatic acrylic monomer of formula (I):
A is H or CH₃;
B is O;
D is O or nothing;
E is CH₃ or H;
x is 2 or 3, provided that not more than one CHE group has E=CH₃;
y is 0; and
z is 0-2, provided that if D=nothing, then z/0.

Clause 4. The ophthalmic device material of any of Clauses 1 to 3, wherein for the cycloaliphatic acrylic monomer of formula (I):
A is H or CH₃;
B is O;
D is nothing;
E is H;
x is 3;
y is 0; and
z is 2.

Clause 5. The ophthalmic device material of any of Clauses 1 to 4, wherein the cycloaliphatic acrylic monomer of formula (I) is selected from the group consisting of: 2-cyclohexylethyl acrylate; 2-cyclopentylethyl acrylate; 3-cyclohexylpropyl acrylate; 3-cyclopentylpropyl acrylate; and 2-(cyclohexyloxy)ethyl acrylate.

Clause 6. The ophthalmic device material of any of Clauses 1 to 5, wherein the mixture comprises a total of 75-85 wt. % of cycloaliphatic acrylic monomer of formula (I).

Clause 7. The ophthalmic device material of any of Clauses 1 to 6, wherein the mixture comprises a total of 77-82 wt. % of cycloaliphatic acrylic monomer of formula (I).

Clause 8. The ophthalmic device material of any of Clauses 1 to 7, wherein said second hydrophobic monomer is aliphatic acrylic monomer of formula (II):

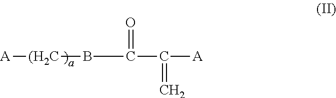

wherein: A is H or CH₃;
B is O, NR, or S;
and a is 2-12.

Clause 9. The ophthalmic device material of any of Clauses 1 to 8, wherein for the aliphatic acrylic monomer of formula (II):
A is H or CH₃;
B is O; and
a is 4-10.

Clause 10. The ophthalmic device material of any of Clauses 1 to 9, wherein the aliphatic acrylic monomer of formula (II) is selected from the group consisting of: n-butyl acrylate (BA), n-hexyl acrylate (HA), n-octyl acrylate (nOA), and/or n-dodecyl acrylate (DDA). Most preferred is n-octyl acrylate (nOA).

Clause 11. The ophthalmic device material of any of Clauses 1 to 10, wherein the cross-linking agent is selected from the group consisting of: 1,4-butanediol diacrylate; triethylene glycol diacrylate; neopentyl glycol diacrylate; decyl diacrylate; and tricyclodecane dimethanol diacrylate.

Clause 12. The ophthalmic device material of any of Clauses 1 to 11, wherein the mixture comprises 0.1-5 wt. % of a reactive UV absorber.

Clause 13. The ophthalmic device material of any of Clauses 1 to 12, wherein the reactive UV absorber includes one or more of 2-(2′-hydroxy-3′-methallyl-5′-methylphenyl)benzotriazole, 3-(2H-benzo[d][1,2,3]triazol-2-yl)-4-hydroxyphenylethyl methacrylate, or 2-(3-(tert-butyl)-4-hydroxy-5-(5-methoxy-2H-benzo[d][1,2,3]triazol-2-yl)phenoxy)ethyl methacrylate.

Clause 14. An ophthalmic device material made by polymerizing a mixture of monomers wherein the mixture comprises:
a) 2-cyclohexylethyl acrylate;
b) 2-hydroxethyl methacrylate;
c) N-Octyl acrylate; and
d) a cross-linking agent having a molecular weight of 100-500 Daltons;
wherein the ophthalmic device material has a refractive index of 1.48-1.50, an Abbe number greater than 52, a Tg<15 °C, and an equilibrium water content less than 2%.

Clause 15. The ophthalmic device material of Clause 14, made by polymerizing a mixture of monomers wherein the mixture comprises:
a) 65-91 wt. % 2-cyclohexylethyl acrylate;
b) 10-25 wt. % 2-hydroxyethyl methacrylate;
c) 3-17 wt. % N-Octyl acrylate; and
d) 1-2.2 wt. % neopentyl glycol diacrylate;
wherein the ophthalmic device material has a refractive index of 1.48-1.50, an Abbe number greater than 52, a Tg<10 °C, and an equilibrium water content less than 2%.

Clause 16. The ophthalmic device material of Clauses 14 or 15, wherein the mixture comprises 0.1-5 wt. % of a reactive UV absorber.

Clause 17. The ophthalmic device material of any of Clauses 14 to 16, wherein the mixture comprises 0.0051-1 wt. % of a reactive blue-light absorbing compound.

Clause 18. The ophthalmic device material of any of Clauses 14 to 17, wherein the reactive blue-light absorbing compound includes N-2-[3-(2′-methylphenylazo)-4-hydroxyphenyl]ethyl methacrylamide.

Clause 19. An ophthalmic device material made by polymerizing a mixture of monomers wherein the mixture comprises:
a) 65-91 wt. % 2-cyclohexylethyl acrylate;
b) 10-25 wt. % 2-hydroxyethyl methacrylate;
c) 3-17 wt. % N-Octyl acrylate; and
d) 1-2.2 wt. % neopentyl glycol diacrylate;
wherein the ophthalmic device material has a refractive index of 1.48-1.50, an Abbe number greater than 50, and a Tg<10 °C.

Clause 20. The ophthalmic device material of Clause 19, wherein the equilibrium water content of the polymerized ophthalmic device material is less than 2%.

As used herein, the word “exemplary” means “serving as an example, instance, or illustration.” The embodiments described herein are not limiting, but rather are exemplary only. It should be understood that the described embodiments are not necessarily to be construed as preferred or advantageous over other embodiments. Moreover, the term “embodiments” does not require that all disclosed embodiments include the discussed feature, advantage, or mode of operation.

As utilized herein, “and/or” means any one or more of the items in the list joined by “and/or.” As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y and z.” As utilized herein, the term “exemplary” means serving as a non-limiting example, instance, or illustration. As utilized herein, the terms “e.g.” and “for example” set off lists of one or more non-limiting examples, instances, or illustrations.

The foregoing description and accompanying figures illustrate the principles, preferred embodiments, and modes of operation. However, the disclosure should not be construed as being limited to the particular embodiments discussed above. Additional variations of the embodiments discussed above will be appreciated by those skilled in the art.

While the present methods and/or materials have been described with reference to certain implementations, it will be understood by those skilled in the art that various changes may be made and equivalents may be substituted without departing from the scope of the present methods and/or materials. In addition, many modifications may be made to adapt a particular situation or material to the teachings of the present disclosure without departing from its scope. For example, block and/or components of disclosed examples may be combined, divided, re-arranged, and/or otherwise modified. Therefore, the present methods and/or materials are not limited to the particular implementations disclosed. Instead, the present methods and/or materials will include all implementations falling within the scope of the appended claims, both literally and under the doctrine of equivalents. While the methods and materials are described as being employed in connection with optical lenses, such as IOLs, the disclosed teachings may be similarly applied to other systems and applications.

All documents cited herein, including journal articles or abstracts, published or corresponding U.S. or foreign patent applications, issued or foreign patents, or any other documents are each entirely incorporated by reference herein, including all data, tables, figures, and text presented in the cited documents.

What is claimed is:

1. An ophthalmic device material made by polymerizing a mixture of monomers wherein the mixture comprises:
a) 55-85 wt. % of a first hydrophobic monomer;
b) 10-22 wt. % of a hydrophilic monomer;
c) 2-18 wt. % of a second hydrophobic monomer; and
d) 1.0-4.0 wt. % of a cross-linking agent having a molecular weight of 100-500 Daltons;
wherein the ophthalmic device material has a refractive index of 1.48-1.50, an Abbe number greater than 52, a Tg<15 °C, and an equilibrium water content less than 2%.

2. The ophthalmic device material of claim 1, wherein said first hydrophobic monomer is of cycloaliphatic acrylic monomer of formula (I): 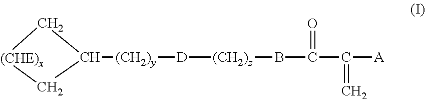
wherein: A is H or CH₃;
B is O, NR, or S;
D is O, S, or nothing (i.e., a single bond);
Eis CH₃, CH₂CH₃, CH(CH₃)₂, C(CH₃)₃, CH₂OH or H;
R is H, CH₃, CH₂CH₃, or CH(CH₃)₂;
x is 1-4, provided that if x is >1, then not more than one CHE group has EH;
y is 0-2; and
z is 0-4, provided that if D=nothing, then z/0.

3. The ophthalmic device material of claim 2 wherein for the cycloaliphatic acrylic monomer of formula (I):
A is H or CH₃;
B is O;
D is O or nothing;
E is CH₃ or H;
x is 2 or 3, provided that not more than one CHE group has E=CH₃;
y is 0; and
z is 0-2, provided that if D=nothing, then z/0.

4. The ophthalmic device material of claim 2 wherein for the cycloaliphatic acrylic monomer of formula (I):
A is H or CH₃;
B is O;
D is nothing;
E is H;
x is 3;
y is 0; and
z is 2.

5. The ophthalmic device material of claim 2 wherein the cycloaliphatic acrylic monomer of formula (I) is selected from the group consisting of: 2-cyclohexylethyl acrylate; 2-cyclopentylethyl acrylate; 3-cyclohexylpropyl acrylate; 3-cyclopentylpropyl acrylate; and 2-(cyclohexyloxy)ethyl acrylate.

6. The ophthalmic device material of claim 2 wherein the mixture comprises a total of 75-85 wt. % of cycloaliphatic acrylic monomer of formula (I).

7. The ophthalmic device material of claim 5 wherein the mixture comprises a total of 77-82 wt. % of cycloaliphatic acrylic monomer of formula (I).

8. The ophthalmic device material of claim 1, wherein said second hydrophobic monomer is aliphatic acrylic monomer of formula (II): 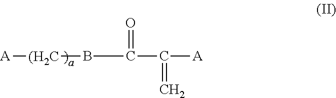
wherein: A is H or CH₃;
B is O, NR, or S;
and a is 2-12.

9. The ophthalmic device material of claim 8, wherein for the aliphatic acrylic monomer of formula (II):
A is H or CH₃;
B is O; and
a is 4-10.

10. The ophthalmic device material of claim 8, wherein the aliphatic acrylic monomer of formula (II) is selected from the group consisting of: n-butyl acrylate (BA), n-hexyl acrylate (HA), n-octyl acrylate (nOA), and/or n-dodecyl acrylate (DDA).

11. The ophthalmic device material of claim 1 wherein the cross-linking agent is selected from the group consisting of: 1,4-butanediol diacrylate; triethylene glycol diacrylate; neopentyl glycol diacrylate; decyl diacrylate; and tricyclodecane dimethanol diacrylate.

12. The ophthalmic device material of claim 1, wherein the mixture comprises 0.1-5 wt. % of a reactive UV absorber.

13. The ophthalmic device material of claim 12, wherein the reactive UV absorber includes one or more of 2-(2′-hydroxy-3′-methallyl-5′-methylphenyl)benzotriazole, 3-(2H-benzo[d][1,2,3]triazol-2-yl)-4-hydroxyphenylethyl methacrylate, or 2-(3-(tert-butyl)-4-hydroxy-5-(5-methoxy-2H-benzo[d][1,2,3]triazol-2-yl)phenoxy)ethyl methacrylate.

14. An ophthalmic device material made by polymerizing a mixture of monomers wherein the mixture comprises:
a) 2-cyclohexylethyl acrylate;
b) 2-hydroxethyl methacrylate;
c) N-Octyl acrylate; and
d) a cross-linking agent having a molecular weight of 100-500 Daltons;
wherein the ophthalmic device material has a refractive index of 1.48-1.50, an Abbe number greater than 52, a Tg<15 °C, and an equilibrium water content less than 2%.

15. The ophthalmic device material of claim 14, made by polymerizing a mixture of monomers wherein the mixture comprises:
a) 65-91 wt. % 2-cyclohexylethyl acrylate;
b) 10-25 wt. % 2-hydroxyethyl methacrylate;
c) 3-17 wt. % N-Octyl acrylate; and
d) 1-2.2 wt. % neopentyl glycol diacrylate;
wherein the ophthalmic device material has a refractive index of 1.48-1.50, an Abbe number greater than 52, a Tg<10 °C, and an equilibrium water content less than 2%.

16. The ophthalmic device material of claim 14, wherein the mixture comprises 0.1-5 wt. % of a reactive UV absorber.

17. The ophthalmic device material of claim 14, wherein the mixture comprises 0.0051-1 wt. % of a reactive blue-light absorbing compound.

18. The ophthalmic device material of claim 17, wherein the reactive blue-light absorbing compound includes N-2-[3-(2′-methylphenylazo)-4-hydroxyphenyl]ethyl methacrylamide.

19. An ophthalmic device material made by polymerizing a mixture of monomers wherein the mixture comprises:
a) 65-91 wt. % 2-cyclohexylethyl acrylate;
b) 10-25 wt. % 2-hydroxyethyl methacrylate;
c) 3-17 wt. % N-Octyl acrylate; and
d) 1-2.2 wt. % neopentyl glycol diacrylate;
wherein the ophthalmic device material has a refractive index of 1.48-1.50, an Abbe number greater than 50, and a Tg<10 °C.

20. The ophthalmic device material of claim 19, wherein the equilibrium water content of the polymerized ophthalmic device material is less than 2%.
